(19) United States
(12) Patent Application Publication
(10) Pub. No.: US 2019/0351156 A1
(43) Pub. Date: Nov. 21, 2019

(54) HIGH TEMPERATURE RESISTANT GLASS COATINGS FOR METALLIC COILS

(71) Applicant: Intrepid Brands, LLC, Louisville, KY (US)

(72) Inventor: Rakesh Guduru, Weston, FL (US)

(21) Appl. No.: 16/414,091

(22) Filed: May 16, 2019

Related U.S. Application Data
(60) Provisional application No. 62/672,207, filed on May 16, 2018.

(51) Int. Cl. A61M 11/04; A24F 47/00; A61M 15/06; A61M 11/00

(52) U.S. Cl. CPC A61M 11/042; A61M 11/005; A61M 15/06; A24F 47/008

(57) ABSTRACT

An atomizer assembly for use with an electronic vaporizer device includes a metallic coil coated with a high temperature resistant glass coating to resist oxidation, corrosion and degradation of the coil at high temperatures. The glass coating may further act as a barrier between the coil and a vapor to minimize metal contamination in the vapor.

### PRIORITY

This application claims priority to U.S. Provisional Patent Application No. 62/672,207, entitled “High Temperature Resistant Glass Coatings for Metallic Coils,” filed on May 16, 2018, the disclosure of which is hereby incorporated by reference herein.

### TECHNICAL FIELD

The disclosure is directed to high temperature resistant glass coatings for metallic coils. The high temperature resistant glass coatings may be used as part of an electronic vaporization device, such as an e-cigarette or personal vaporizer, to vaporize certain materials.

### BACKGROUND

An electronic vaporization device may simulate the feeling of smoking by heating a substance to generate an aerosol, commonly called a “vapor”, that a user inhales. Vaporization provides an alternative to combustion for the delivery and consumption of various substances including, but not limited to liquids, i.e., “E-liquids,” waxes, gels and combinations thereof (singularly, “a vaporizable substance,” collectively, “vaporizable substances”). Non-limiting examples of components of vaporizable substances include glycerin, propylene glycol, flavorings, nicotine, medicaments and combinations thereof. Vaporization may be accomplished using electronic vaporization devices, including, but not limited to, electronic cigarettes, electronic cigars, electronic pipes and electronic vaporizers (singularly “EVD,” collectively, “EVDs”).

While EVDs may reduce consumer exposure to toxins as compared to traditional smoking, there may be a cause for concern relating to consumer exposure to trace metal(s) through vapor inhalation. EVDs typically use resistive heating to vaporize the liquids in an atomizer by passing a high current through a conductor, such as a metallic coil (i.e., nickel, aluminum, silver, chromium, iron, Kanthal, Nichrome, etc.) to produce heat, thereby generating a vapor for inhalation. Such heat and harsh environments in the atomizer may cause the metallic coil to oxidize, degrade, volatilize and/or corrode, contaminating the vapor with trace metal(s).

Thus, in some instances, it may be desirable to minimize the process of oxidation, degradation, volatilization and/or corrosion of the metallic coil of the atomizer. While a variety of atomizers have been made and used, it is believed that no one prior to the inventor has made or used an invention as described herein.

### SUMMARY

The unique solution that addresses the aforementioned problems is a metallic coil coated with a high temperature resistant glass as shown and described herein. Such a metallic coil can be used with an atomizer of an EVD to create a barrier between the metallic coil and the vapor. This may minimize or eliminate the process of oxidation, degradation, volatilization and/or corrosion of the atomizer to thereby limit consumer exposure to one or more trace metals.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a cross-sectional view of a typical Electronic Vaporization Device.

FIG. 2 depicts a cross-sectional view of a chamber of the EVD of FIG. 1.

FIG. 3 depicts a cross-sectional side view of a glass coated metallic coil, which may be used in a typical EVD as shown in FIG. 1.

FIG. 4 depicts a cross-sectional top view of the glass coated metallic coil of FIG. 3.

FIG. 5 depicts a schematic of a method to manufacture the metallic coil of FIG. 3.

FIG. 6 depicts a schematic of a method to coat the metallic coil of FIG. 3.

FIG. 7 depicts a scanning electron microscopy image of the glass coated metallic coil of FIG. 3.

FIG. 8 depicts an electron dispersion spectroscopy image showing the distribution of silicon dioxide on the metallic coil of FIG. 3.

FIG. 9 depicts an electron dispersion spectroscopy image showing the distribution of iron on the metallic coil of FIG. 3.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

All percentages, parts and ratios as used herein, are by weight of the total composition of ambient moisture-activatable surface treatment powder, unless otherwise specified. All such weights, as they pertain to listed ingredients, are based on the active level and, therefore, do not include solvents or by-products that may be included in commercially available materials, unless otherwise specified.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9 and so forth.

All references to singular characteristics or limitations of the present disclosure shall include the corresponding plural characteristic or limitation and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

As used herein, the term “comprising” means that the various components, ingredients, or steps, can be conjointly employed in practicing the present invention. Accordingly, the term “comprising” encompasses the more restrictive terms “consisting essentially of” and “consisting of.”

As used herein, “trace metal” collectively refers to metal, metal alloy and combinations of metal or metal alloy that is present in a vapor in a small, but measurable amount.

As used herein, “substantially free” refers to an amount in a vapor of about 1 wt. % or less, about 0.1 wt. % or less, about 0.01 wt. % or less or 0% (i.e., completely free of), one or more trace metals.

As used herein, “chamber,” “liquid chamber,” “tank,” “liquidmizer,” “cartomizer,” “disposable pod” and “clearomizer,” are used interchangeably to mean a reservoir that contains vaporizable substance to be vaporized by an EVD.

As used herein, “high temperature resistant glass” means glass having a glass transition temperature, “Tg,” of from about 300° C. to about 1,500° C. Tg is measured using ASTM E1640-18: Standard Test Method for Assignment of the Glass Transition Temperature By Dynamic Mechanical Analysis.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

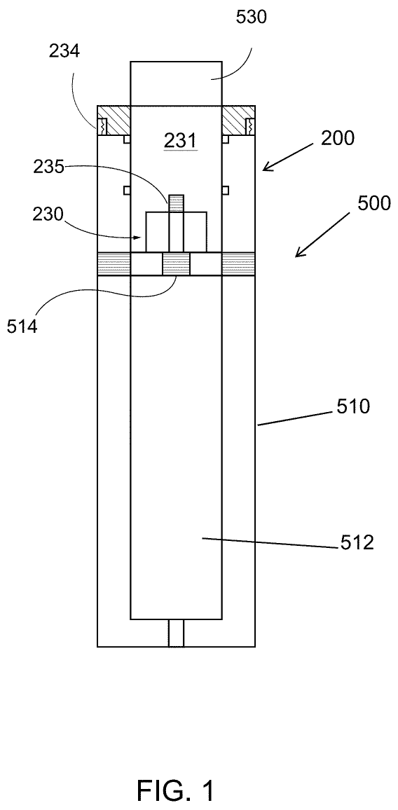

FIG. 1 shows a typical EVD 500 comprising a battery compartment 510 comprising a battery 512 that is removably attached to a chamber 200 by connector 514. The chamber 200 is in turn removeably attached to a mouthpiece 530. The chamber may be filled with a vaporizable substance through its open top, i.e., be a “top-filled chamber,” or it may be filled with a vaporizable substance through its open bottom, i.e., a “bottom-filled chamber.” As is known in the art, some EVDs comprise a battery compartment that is permanently affixed to a chamber of an EVD.

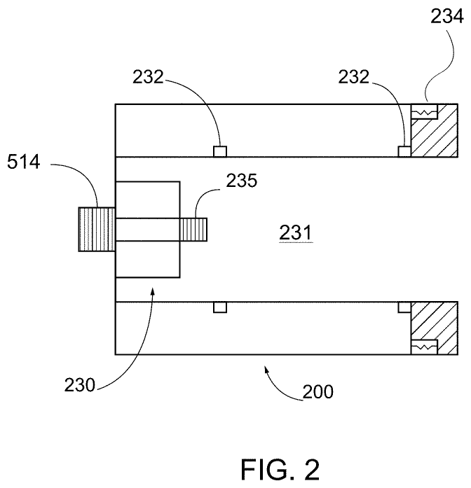

FIG. 2 shows the chamber 200 of FIG. 1 comprising an atomizer assembly 230. As illustrated, the atomizer assembly 230 comprises a metallic coil 235. This metallic coil 235 can be wrapped within an absorbent wick material such that the metallic coil 235 is positioned within the absorbent wick material. In some other versions, the absorbent wick material can be inserted through the metallic coil 235 such that the metallic coil 235 is positioned about the absorbent wick material. Exemplary wick material of use may be selected from cotton, nylon, porous ceramic and combinations thereof.

Extending from the atomizer assembly 230 is a vapor chimney 231, which is surrounded in part by a silicone or rubber ring 232. When the chamber 200 is assembled, the atomizer assembly 230 and vapor chimney 231 fit into the chamber 200. The chamber 200 is capped at its open top by a hollow metal ring 234 that is threaded on the inside and which serves as the attachment point of the mouthpiece to the chamber 200.

Accordingly, the metallic coil 235 of the atomizer assembly 230 becomes hot when supplied with electricity from the battery compartment 510 due to its resistance to the flow of electric current. The wick material in turn acts to transport the vaporizable substance, i.e., the E-liquid, gel or melted wax, to the metallic coil 235 to heat it and release vapor. The resulting vapor may then pass through the vapor chimney 231 to be delivered to the consumer via the mouthpiece 530.

Because heat and harsh environments in the atomizer assembly 230 may cause the metallic coil 235 to oxidize, degrade, volatilize and/or corrode, the resulting vapor may be contaminated with one or more trace metals. Surprisingly, the metallic coil 235 can be coated with a high temperature resistant glass and used with an atomizer of an EVD to create a barrier between the metallic coil and the vapor to minimize the process of oxidation, degradation and/or corrosion of the atomizer assembly to thereby limit the exposure to one or more trace metals while still providing sufficient heat to vaporize vaporizable substances and providing vapor that is substantially free of one or more trace metals.

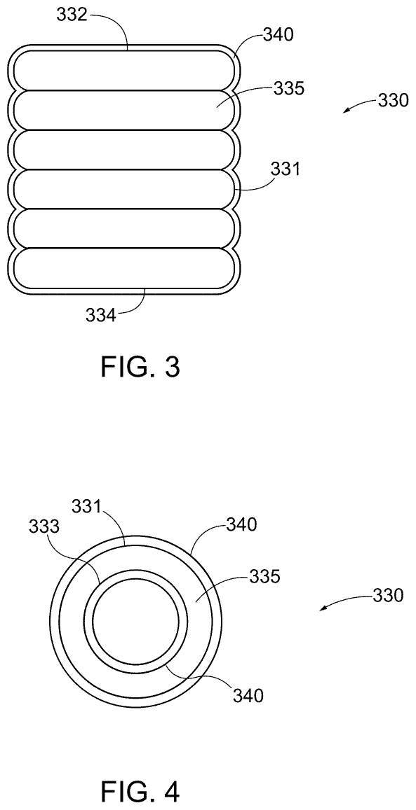

A first exemplary coated coil 330 is shown in FIGS. 3-4. As shown, the coil 330 comprises a metal wrapped to form a coil 335 having a top surface 332, a bottom surface 334, an outer side surface 331 and an inner side surface 333. Coil 335 may comprise any suitable metal. For example, coil 335 may comprise metal selected from the group of nickel, aluminum, silver, chromium, iron, an alloy of FeCrAl (e.g., Kanthal® which is an alloy comprising 20-30 wt % Cr, 20-30 wt % Al and the balance Fe (Sandvik Group, Sweden), nichrome (an alloy of nickel with chromium (at 10-20 wt %) and sometimes iron (up to 25 wt %), platinum, stainless steel, titanium and combinations thereof. The metallic coil 335 may be coated with a coating 340 comprising a high temperature resistant glass. For example, coating 340 may comprise glass selected from the group of silicon dioxide, graphene, NaAlSi₃O₈, NaAlSi₂O6, NaAlSiO₄ and KAlSi₃O₈ and combinations thereof. Coating 340 thereby protects the coil 335 from the heat and harsh condition in an atomizer assembly of an EVD. For instance, the atomizer assembly may routinely reach temperatures of from about 150° C. to about 600° C., from about 180° C. to about 300° C. or from about 150° C. to about 180° C. Further, the atomizer assembly may routinely reach a temperature of about 180° C. or about 200° C. Accordingly, the coating 340 is designed to allow thermal conduction by the coil 335 to reach the temperatures required to vaporize the material in the chamber 200, while resisting oxidation, corrosion, volatilization and/or degradation of the coil 335. One of skill in the art may determine the appropriate glass to use for the coating by taking into account the Tg of the glass and comparing it to the anticipated temperature that the resulting atomizer assembly may reach to thus ensure that the glass coating remains in a “glassy state,” i.e., remains intact, when vaporizing the vaporizable substance. The Tg of a material is the temperature that when exceeded causes the material to change from a rigid amorphous state to a viscous elastic state. “Heat resistant glass” as used herein means glass that resists heat below its Tg without changing its physical state.

Coating 340 may have any suitable thickness so as to provide for sufficient heat transfer from the coil 335 through the coating 340 to the vaporizable substance. One of skill in the art may determine the appropriate thickness to use by taking into account the heat transfer coefficient of the glass from which the coating is made. Exemplary coatings of use may have a thickness of from about 0.1 microns to about 1,000 microns or from about 0.1 microns to about 0.7 microns. Coating 340 covers all or portion(s) of the coil 335. How much of and/or where the coil 335 comprises coating 340, may be determined by one skilled in the art by taking into account which portion(s) of coil 335 will be exposed to a vaporizable substance and/or vapor during use. FIGS. 3-4, show exemplary coating 340 that covers an entire outer surface of the coil 335, such that the coating 340 covers the top surface 332, bottom surface 334, outer side surface 331 and inner side surface 333 of the coil 335. Further exemplary coatings 340 may only partially cover coil 335, for example, from about 50% to about 90% of the outer surface of the coil. In these exemplary embodiments, it is understood that those portions of the outer surface of the coil that are covered by coating 340 are those portions of the coil that may be exposed to a vaporizable substance and/or vapor during use.

While the example shown in FIGS. 3-4 shows the coating 340 as having a uniform thickness, the thickness of the coating 340 may also be variable across the outer surface of the coil 335. The coating 340 may thereby act as a barrier between the metal of the coil 335 and the vapor, reducing or eliminating any metal contamination of the vapor. The coating 340 may further exhibit high shear stress, which may allow the consumer a higher degree of flexibility during consumer installation of coil 335 in the atomizer assembly 230. Of course, other suitable configurations for the coated coil 330 will be apparent to one of ordinary skill in the art in view of the teachings herein.

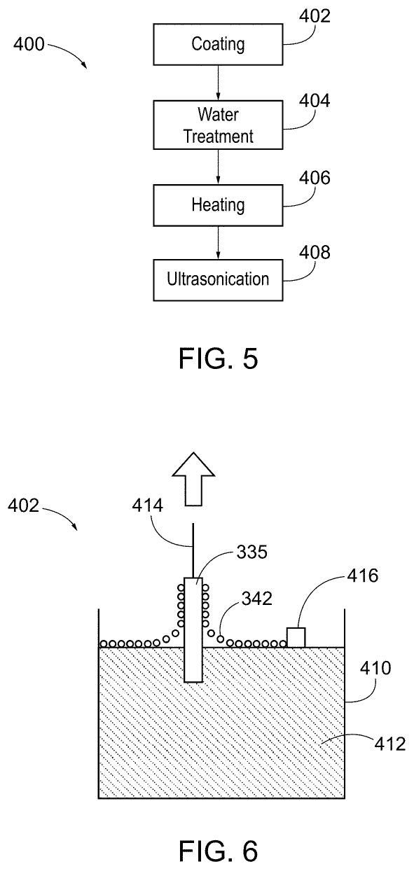

An exemplary method 400 of making a coated metallic coil 330 is shown in FIG. 5. Method 400 comprises a coating step 402, a water treatment step 404, a heating step 406 and an ultrasonication step 408. In the coating step 402, the metallic coil 335 may be dipped in a tetraethyl orthosilicate (TEOS) or other suitable solution using a Languir-Blodgett method either before or after the coil 335 is formed into a coil-shape. For instance, as shown in FIG. 6, the coil 335 may be held by a support 414 and dipped into a trough 410 containing the TEOS solution 412 such that a single monolayer 342 or multiple monolayers of the TEOS solution 412 is transferred to the coil 335. In some instances, the TEOS molecules on the surface of the solution 412 may be compressed and/or expanded by a barrier 416. The use of a barrier may allow for a high degree of control over the thickness and/or density of the coating 340. Once the coil 335 is coated, it may be treated with water, such as by surface hydrolysis, to convert the TEOS into a glass coating comprising silicon dioxide. To further enhance the silicon dioxide formation, the coil 335 may be subjected to a high temperature, such as about 600° C. in a furnace, for about 30 minutes. The coil 335 may then be subjected to ultrasonication to remove excess glass from the coil 335, leaving behind an ultra-thin layer of glass coating 340.

A further exemplary method of making a coated metallic coil 330 may comprise thermally forming one or more oxide layers on an outer surface of the coil 335. Exemplary method may comprise applying silicon to an outer surface of the coil 335, heating the silicon to a temperature of from about 800° C. to about 1,200° C. and forming a coating comprising silicon dioxide on metallic coil 330.

Still other suitable methods for coating the coil will be apparent to one with ordinary skill in the art in view of the teachings herein.

For instance, the coil 335 may be coated in an extrusion coating method while the coating material is hot, followed by annealing. Thus, the coated metallic coils may exhibit a high resistance to oxidation, corrosion, volatilization and/or degradation. The coil coated with a high temperature resistant glass coating is also operable to vaporize a vaporizable substance within an electronic vaporization device to provide a vapor that is substantially free from one or more trace metals. Exemplary trace metals may be selected from nickel, aluminum, silver, chromium, iron, an alloy of FeCrAl (e.g., Kanthal® which is an alloy comprising 20-30 wt % Cr, 20-30 wt % Al and the balance Fe (Sandvik Group, Sweden), nichrome (an alloy of nickel with chromium (at 10-20 wt %) and sometimes iron (up to 25 wt %), platinum, stainless steel, titanium and combinations thereof.

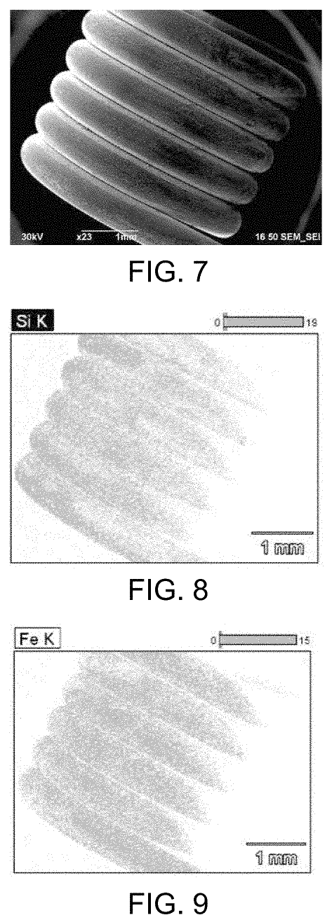

Referring to FIG. 7, a scanning electron microscopy image of an exemplary metal coil coated with glass 330 is shown. A distribution of glass using the TEOS precursor is shown using electron dispersion spectroscopy in FIG. 8. For a comparison, an iron (Fe) distribution in the coil 330 is shown in FIG. 9. As can be seen by FIGS. 7-9, the coating 340 has a substantially even distribution on the surface of the coil 330.

Having shown and described various versions of the present invention, further adaptations of the methods and systems described herein may be accomplished by appropriate modifications by one of ordinary skill in the art without departing from the scope of the present invention. Several of such potential modifications have been mentioned and others will be apparent to those skilled in the art. For instance, the examples, versions, geometrics, materials, dimensions, ratios, steps and the like discussed above are illustrative and are not required. Accordingly, the scope of the present invention should be considered in terms of the following claims and is understood not to be limited to the details of structure and operation shown and described in the specification and drawings.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An atomizer assembly comprising a metallic coil, wherein an outer surface of the coil is coated with a glass coating, wherein the glass coating is configured to resist oxidation, corrosion, volatilization and degradation of the coil.

### EXAMPLE 2

An atomizer assembly according to example 1 or any of the following examples up to example 11, wherein the coating comprises glass selected from silicon dioxide, graphene, NaAlSi₃O₈, NaAlSi₂O₆, NaAlSiO₄ and KAlSi₃O₈ and combinations thereof.

### EXAMPLE 3

An atomizer assembly according to either one of the preceding examples or following examples up to example 11, wherein the coating is heat resistant at temperatures of from about 150° C. to about 600° C.

### EXAMPLE 4

An atomizer assembly according to any one of the preceding examples or following examples up to example 11, wherein the coating has a thickness of from about 0.1 microns to about 1,000 microns, or from about 0.1 microns to about 0.7 microns.

### EXAMPLE 5

An atomizer assembly according to example 4, or any one of the following examples up to example 11, wherein the thickness of the coating is uniform across the outer surface of the coil.

### EXAMPLE 6

An atomizer assembly according to examples 1 through 4, or any one of the following examples up to example 11, wherein the thickness of the coating varies across the outer surface of the coil.

### EXAMPLE 7

An atomizer assembly according to any one of the preceding examples or following examples up to example 11, wherein the outer surface of the coil is entirely coated by the coating.

### EXAMPLE 8

An atomizer assembly according to any one of the preceding examples with the exception of example 7, or according to any one of the following examples up to example 11, wherein a portion of the outer surface of the coil is coated by the coating, for example, wherein at least about 95% of the outer surface of the coil is coated by the glass coating or wherein from about 50% to about 95% of the outer surface of the coil is coated by the glass coating.

### EXAMPLE 8a

An atomizer assembly according to any one of the preceding examples with the exception of examples 7 and 8, or according to any one of the following examples up to example 11, wherein only a portion or portions of the outer surface of the coil is/are coated by the coating, for example, wherein less than about 95% of the outer surface of the coil is coated by the glass coating or wherein less than from about 50% to about 95% of the outer surface of the coil is coated by the glass coating.

### EXAMPLE 9

An atomizer assembly according to any one of the preceding examples or following examples up to example 11, wherein the coating is configured as a barrier between the coil and a vapor to minimize trace metal contamination in the vapor.

### EXAMPLE 10

An atomizer assembly according to any one of the preceding examples or following examples up to example 11, wherein the coating comprises a high shear stress.

### EXAMPLE 11

An atomizer assembly according to any one of the preceding examples, wherein the coating comprises thermal conductive properties to allow the coil to be heated to temperatures of from about 150° C. to about 600° C.

### EXAMPLE 12

An electronic vaporizer device comprising:
a chamber configured to hold a vaporizable substance;
an atomizer assembly according to any one of examples 1 through 12, the atomizer assembly further comprising a wick material coupled with the metallic coil, wherein the wick material is configured to transport the vaporizable substance to the metallic coil; and
a battery compartment configured to supply a current to the metallic coil to thereby heat the metallic coil such that the vaporizable substance transported to the metallic coil is vaporized to form a vapor;
wherein the metallic coil is coated with glass coating, for example a high temperature resistant glass coating having a glass transition temperature (Tg) of from about 300° C. to about 1,500° C., wherein the coating is configured to resist oxidation, corrosion, volatilization, and degradation of the metallic coil.

### EXAMPLE 12a

An electronic vaporizer device comprising:
a chamber configured to hold a vaporizable substance;
an atomizer assembly comprising a wick material coupled with a metallic coil, wherein the wick material is configured to transport the vaporizable substance to the metallic coil; and
a battery compartment configured to supply a current to the metallic coil to thereby heat the metallic coil such that the vaporizable substance transported to the metallic coil is vaporized to form a vapor;
wherein the metallic coil is coated with glass coating, for example a high temperature resistant glass coating having a glass transition temperature (Tg) of from about 300° C. to about 1,500° C., wherein the coating is configured to resist oxidation, corrosion, volatilization, and degradation of the metallic coil.

### EXAMPLE 13

A device according to example 12 or 12a or any of the following examples up to example 15, wherein the coating comprises silicon dioxide, graphene, NaAlSi₃O₈, NaAlSi₂O6, NaAlSiO₄ and KAlSi₃O₈ and combinations thereof.

### EXAMPLE 14

A device according to any one of examples 12, 12a or 13 and any of the following examples up to example 15, wherein the coating has a thickness of from about 0.1 microns to about 1,000 microns or from about 0.1 microns to about 0.7 microns.

### EXAMPLE 15

A device according to any one of examples 12, 12a, 13, or 14, wherein the coating is configured as a barrier between the coil and the vapor to minimize metal contamination in the vapor.

### EXAMPLE 16

A method of making a coated metallic coil for use with an electronic vaporization device, the method comprising coating an outer surface of the metallic coil with a glass coating comprising glass having a glass transition temperature (Tg) of from about 300° C. to about 1,500° C.

### EXAMPLE 17

A method according to example 16 or any of the following examples with the exception of example 18, the method comprising: dipping the metallic coil in a solution comprising tetraethyl orthosilicate, transferring one or more monolayers of the tetraethyl orthosilicate to the metallic coil, treating the one or more monolayers of the tetraethyl orthosilicate transferred to the metallic coil with water, heating the metallic coil to convert the tetraethyl orthosilicate solution into silicon dioxide to form the coated metallic coil, wherein the coating comprises silicon dioxide.

### EXAMPLE 18

A method according to example 16 or any one of the following examples, comprising: applying silicon to the outer surface of the coil,

heating the silicon to a temperature of from about 800° C. to about 1,200° C., and

forming a coated metallic coil,

wherein the coating comprises silicon dioxide.

### EXAMPLE 19

A method according to any one of examples 16 through 18 or the following example, wherein the coating has a thickness of from about 0.1 microns to about 1,000 microns, or from about 0.1 microns to about 0.7 microns.

### EXAMPLE 20

A method according to either one of examples 17 or 18, further comprising subjecting the coated metallic coil to ultrasonication to remove excess silicon dioxide from the coil.

### EXAMPLE 21

A method of operating an electronic vaporization device comprising vaporizing a vaporizable substance with a coil coated with a high temperature resistant glass coating, for example a high temperature resistant glass coating having a glass transition temperature (Tg) of from about 300° C. to about 1,500° C., wherein the vapor from the vaporizable substance is substantially free from trace metals.

### EXAMPLE 21a

A method of operating an electronic vaporization device comprising vaporizing a vaporizable substance with a coated metallic coil according to any one of examples 1 through 12, wherein the vapor from the vaporizable substance is substantially free from trace metals.

### EXAMPLE 22

The method according to either one of examples 21 or 21a, wherein the trace metals are selected from nickel, aluminum, silver, chromium, iron, an alloy of FeCrAl (e.g., Kanthal® which is an alloy comprising 20-30 wt % Cr, 20-30 wt % Al and the balance Fe (Sandvik Group, Sweden), nichrome (an alloy of nickel with chromium (at 10-20 wt %) and sometimes iron (up to 25 wt %), platinum, stainless steel, titanium and combinations thereof.

I/we claim:

1. An atomizer assembly comprising a metallic coil, wherein an outer surface of the coil is coated with a glass coating, wherein the glass coating is configured to resist oxidation, corrosion, volatilization and degradation of the coil.

2. The atomizer assembly of claim 1, wherein the glass coating comprises glass having a glass transition temperature (Tg) of from about 300° C. to about 1,500° C.

3. The atomizer assembly of claim 1, wherein the glass coating comprises glass selected from silicon dioxide, graphene, NaAlSi₃O₈, NaAlSi₂O6, NaAlSiO₄ and KAlSi₃O₈ and combinations thereof.

4. The atomizer assembly of claim 1, wherein the glass coating is heat resistant at temperatures of from about 150° C. to about 600° C.

5. The atomizer assembly of claim 1, wherein the glass coating has a thickness of from about 0.1 microns to about 1,000 microns.

6. The atomizer assembly of claim 1, wherein the glass coating has a thickness of from about 0.1 microns to about 0.7 microns.

7. The atomizer assembly of claim 1, wherein the thickness of the glass coating is uniform across the outer surface of the coil.

8. The atomizer assembly of claim 1, wherein the thickness of the coating varies across the outer surface of the coil.

9. The atomizer assembly of claim 1, wherein at least about 95% of the outer surface of the coil is coated by the glass coating.

10. The atomizer assembly of claim 1, wherein from about 50% to about 95% of the outer surface of the coil is coated by the glass coating.

11. The atomizer assembly of claim 1, wherein the glass coating is configured as a barrier between the coil and a vapor to minimize trace metal contamination in the vapor.

12. The atomizer assembly of claim 1, wherein the glass coating comprises thermal conductive properties to allow the coil to be heated to temperatures of from about 150° C. to about 600° C.

13. An electronic vaporizer device comprising:
a chamber configured to hold a vaporizable substance;
an atomizer assembly comprising a wick material coupled with a metallic coil, wherein the wick material is configured to transport the vaporizable substance to the metallic coil; and
a battery compartment configured to supply a current to the metallic coil to thereby heat the metallic coil such that the vaporizable substance transported to the metallic coil is vaporized to form a vapor;
wherein the metallic coil is coated with a glass coating configured to resist oxidation, corrosion, volatilization and degradation of the metallic coil.

14. The device of claim 13, wherein the glass coating comprises glass selected from silicon dioxide, graphene, NaAlSi₃O₈, NaAlSi₂O6, NaAlSiO₄ and KAlSi₃O₈ and combinations thereof.

15. The device of claim 13, wherein a thickness of the glass coating is uniform across the outer surface of the coil.

16. The device of claim 13, wherein the outer surface of the coil is entirely coated by the glass coating.

17. The device of claim 13, wherein the glass coating is configured as a barrier between the coil and the vapor to minimize metal contamination in the vapor.

18. A method of making a coated metallic coil for use with an electronic vaporization device, the method comprising coating an outer surface of the metallic coil with a glass coating.

19. The method of claim 18, comprising:
dipping the metallic coil in a solution comprising tetraethyl orthosilicate,
transferring one or more monolayers of the tetraethyl orthosilicate to the metallic coil,
treating the one or more monolayers of the tetraethyl orthosilicate transferred to the metallic coil with water, and
heating the metallic coil to convert the tetraethyl orthosilicate solution into silicon dioxide coating to form the coated metallic coil,
wherein the coating comprises silicon dioxide.

20. The method of claim 18, comprising:
applying silicon to the outer surface of the coil,
heating the silicon to a temperature of from about 800° C. to about 1,200° C., and forming the coating,
wherein the coating comprises silicon dioxide.

21. A method vaporizing a vaporizable substance with a metallic coil, wherein an outer surface of the metallic coil is at least partially coated with a glass coating and further wherein the glass coating is configured to resist oxidation, corrosion, volatilization and degradation of the coil, the method comprising:
heating a vaporizable substance with the metallic coil, and
generating a vapor from the vaporizable substance,
wherein the vapor is substantially free from one or more trace metals.

22. The method of claim 20, wherein the one or more trace metals are selected from nickel, aluminum, silver, chromium, iron, an alloy of FeCrAl, nichrome, platinum, stainless steel, titanium and combinations thereof.
